(19) United States
(12) Patent Application Publication
(10) Pub. No.: US 2008/0146576 A1
(43) Pub. Date: Jun. 19, 2008

(54) N-(1-Phthalazin-1-ylpiperidin-4-yl)amides as EP2 receptor modulators

(76) Inventors: Nico Braeuer, Jena (DE); Bernd Buchmann, Hohen Neuendorf (DE); Koppitz Marcus, Berlin (DE); Antonius Ter Laak, Berlin (DE); Gernot Langer, Falkensee (DE); Bernard Lindenthal, Berlin (DE); Olaf Peters, Tabarz (DE); Tim Wintermantel, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/896,923

(22) Filed: Sep. 6, 2007

Related U.S. Application Data
(60) Provisional application No. 60/842,679, filed on Sep. 7, 2006.

(30) Foreign Application Priority Data
Sep. 7, 2006 (EP) 06 090 159.2

(51) Int. Cl. A61K 31/502; C07D 237/30; A61P 15/08; A61P 35/00; A61P 19/10

(52) U.S. Cl. 514/248; 544/237

(57) ABSTRACT

The present invention relates to phthalazin-1-ylpiperidin-4-ylamides of the general formula I, to processes for their preparation and to their use for producing pharmaceutical compositions for treatment of disorders and indications connected to the EP₂ receptor.

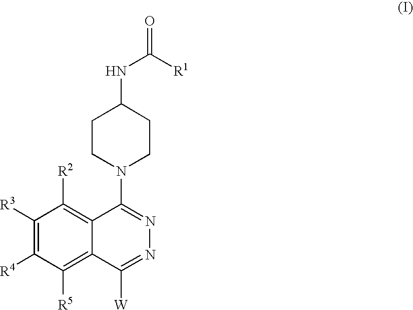

This application claims the benefit of the filing date of U.S. Provisional Application Ser. No. 60/842,679 filed Sep. 7, 2006.

The present invention relates to N-(1-phthalazin-1-ylpiperidin-4-yl)amides as EP₂ receptor modulators, to processes for their preparation and to their use as medicaments.

It has long been known that prostaglandins are the key molecules in the processes of female reproductive biology, such as the regulation of ovulation, of fertilization, of nidation, of decidualization (e.g. placentation) and of menstruation. Prostaglandins also play an important role in pathological changes in the reproductive tract, including menorrhagia, dysmenorrhea, endometriosis and cancer. So far the mechanism by which prostaglandins bring about these changes has not been fully elucidated. Recent findings indicate that prostaglandins, their receptors and the signal transduction pathways thereof are involved in processes such as angiogenesis, apoptosis, proliferation and in inflammatory/anti-inflammatory and immunological processes.

The effects of the prostaglandins are mediated by their G-protein-coupled receptors, which are located on the cell surface. Prostaglandin E₂ (PGE₂) is of particular interest, as it achieves extremely varied cellular effects by binding to functionally different receptor subtypes, namely the EP₁, EP₂, EP₃ and EP₄ receptors. The receptor subtypes to which prostaglandin E₂ binds appear to be of particular interest for the receptor-mediated effects that play a role in fertility regulation. Thus, it has been shown that the reproductive functions in EP₂ knock-out mice (EP₂^{-/-}), i.e. mice that no longer carry the PGE₂ receptor subtype EP₂, are affected adversely, and that these animals have a smaller litter size (Matsumoto et al., 2001, Biology of Reproduction 64, 1557-1565). It has also been shown that these EP₂ knock-out mice (Hizaki et al. Proc Natl Acad Sci U.S.A. 1999 Aug. 31; 96(18), 10501-10506) have markedly reduced cumulus expansion and pronounced subfertility, which demonstrates the significance of the prostaglandin EP₂ receptor for this process. The EP₂ receptor is accordingly an important target for the development of medicinal products for the regulation of female fertility. The existence of 4 subclasses of the PGE₂ receptor offers the possibility of targeted development of compounds with selective action. At present, however, hardly any selective EP₂ receptor ligands that bind to the EP₂ subtypes of the PGE₂ receptor are known, as most of the known compounds also bind to the other PGE₂ receptor subtypes, for example the EP₄ receptor.

EP₂ receptor antagonists are described for example in application US2005059742 (Jabbour, Medical Research Council). A method is claimed in which an EP₂ and/or an EP₄ antagonist can be used for the treatment of menorrhagia and dysmenorrhea. AH6809 is disclosed as an antagonist of the EP₂ or EP₄ receptor; no other specific antagonists and no new compounds are disclosed.

In an earlier application of the same group (EP 1467738), EP₂ or EP₄ antagonists are claimed for the treatment of pathological states, such as uterine carcinoma, myoma and endometriosis. Again, no new compounds are disclosed.

Ono Pharmaceutical claims, in application WO03/016254, the production of benzene acid or saturated carboxylic acid derivatives, which are substituted with aryl or heterocycles, among other things as PGE₂ receptor antagonists. The disclosed compounds are claimed for the treatment of a large number of diseases, including allergic diseases, Alzheimer's disease, pain, miscarriage, menstrual pains, menorrhagia and dysmenorrhea, endometriosis, bone diseases, ischemia etc. However, the compounds described are characterized by especially high affinity for the EP₃ receptor. In another application (WO04/032964), novel compounds are described, which are also characterized by particularly high affinity for the EP₃ receptor, and also find application as EP₂ antagonists, for the treatment and prophylaxis of allergic diseases.

Application WO04/39807 to Merck Frosst, Canada, discloses the production of pyridopyrrolizines and pyridoindolizines. These compounds are, however, characterized by good binding to the PGD₂ receptor, this receptor being another subtype of the prostaglandin receptor.

Naphthalene derivatives are disclosed as EP₄ receptor ligands by the SmithKline Beecham Corporation in application US2004102508. The claimed compounds find application for the treatment or prophylaxis of pain, allergic reactions and neurodegenerative diseases.

EP₄ antagonists (γ-lactams) are claimed in application WO03/103604 (Applied Research Systems). The compounds bind approx. 60-times better to the EP₄ receptor than to the EP₂ receptor and are claimed among other things for the treatment of premature labor, dysmenorrhea, asthma, infertility or fertility disorders. The same company claims, in applications WO03/053923 (substituted pyrrolidines) or WO03/035064 (substituted pyrazolidiones), compounds for the treatment of diseases that are associated with prostaglandins, for example infertility, hypertension and osteoporosis. The compounds bind to the EP₄ and the EP₂ receptor subtypes. Application WO03/037433 claims ω-cycloalkyl, 17 heteroaryl-prostaglandin derivatives as EP₂ receptor antagonists, in particular for the treatment of raised intraocular pressure.

Application WO03/064391 (Pfizer Products) describes metabolites of [3-[[N-(4-tert-butylbenzyl)(pyridin-3-ylsulfonyl)amino]methyl]acetic acid, which inhibit the binding of [³H] prostaglandin-E₂ to the EP₂ receptor. The use of these metabolites for the treatment of osteoporosis is disclosed.

Tani et al. claim, in application US2005124577, 8-azaprostaglandin derivatives for the treatment of immunologic diseases, allergic diseases, premature labor, miscarriage etc. The compounds bind to the EP₂ and EP₄ receptors.

European patent EP 1306087 describes EP₂ receptor agonists, which find application in the treatment of erectile dysfunction. The same structural class is described in European patent EP 860430, which claims use thereof for the production of a medicinal product for the treatment of immunologic diseases, asthma and miscarriage. Application WO04/32965 describes EP₂ receptor agonists that are used for the treatment and prevention of diseases resulting from organ failure due to ischemia. WO04/009117 describes EP₂ and EP₄ receptor agonists for the treatment of diseases caused by uterine contraction, for example menstrual pains.

Applications WO 03/74483 and WO03/09872 describe agonists that bind equally to the EP₂ receptor and to the EP₄ receptor (Ono Pharmaceuticals).

The agonists of the EP and of the EP₄ receptor are often described in connection with the treatment of osteoporosis (WO99/19300, US2003/0166631, WO03/77910, WO03/45371, WO 03/74483 and WO03/09872) and for the treatment of glaucoma (WO04/37813, WO04/37786, WO04/19938, WO03/103772, WO03/103664, U.S. Pat. No. 6,747,037, U.S. Pat. No. 6,410,591, WO03/40123, WO03/47513, WO03/47417).

In patent application WO04/12656, EP₂ receptor agonists are claimed in connection with inflammation.

In patent application WO03/77919, EP₄ receptor agonists are claimed for the treatment of fertility.

To date, however, there are no known selective EP₂ receptor agonists and antagonists which regulate the processes that are ultimately responsible for nidation and decidualization and thus contribute to the promotion or inhibition of fertility.

This leads to the problem of providing stable and effective compounds that bind selectively to the EP₂ receptor, for the development of new medicaments.

It has now been found that, surprisingly, compounds of the general formula I

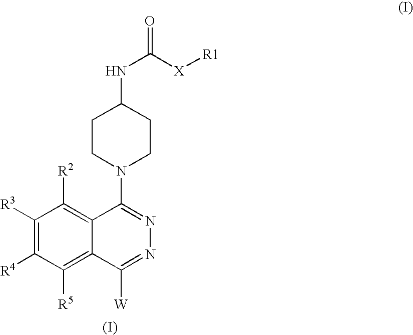

where
W is hydrogen or a C₁-C₄-alkyl group,
X is a (CH₂)ₙ group where n=0-4, a C₂-C₄-alkenyl group, a C₂-C₄-alkynyl group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
   a C₁-C₆-alkyl group which is unsubstituted or optionally substituted,
   a C₃-C₁₀-cycloalkyl radical which may be unsubstituted or optionally substituted,
   an 8-12-membered fused (hetero)arylcycloalkyl radical which is unsubstituted or optionally substituted,
R²-R⁵ are each independently hydrogen, halogen, cyano, or an OR⁶, OC(O)R⁶, S(O)ₙR⁶ where n=0, 1, 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
   a C₁-C₆-alkyl group which may be unsubstituted or optionally substituted,
   a C₃-C₁₀-cycloalkyl ring which may be unsubstituted or optionally substituted,
   a C₂-C₆-alkenyl or C₂-C₆-alkynyl group which may be unsubstituted or optionally substituted,
   a 5-12-membered mono- or bicyclic aryl or heteroaryl ring which may be unsubstituted or optionally substituted,
R⁶, R⁷ are each hydrogen, a C₁-C₆-alkyl group, a C₃-C₁₀-cycloalkyl ring, a 5-12-membered mono- or bicyclic aryl or heteroaryl ring, where the alkyl, cycloalkyl and (het)aryl groups may be unsubstituted or optionally substituted, or
R⁶, R⁷ together form a 3-8-membered ring,
   and the isomers, salts and the cyclodextrin clathrates thereof, overcome the known disadvantages and are able to achieve a better selectivity for the EP₂ receptor and hence a better efficacy and longer action time.

The saturated, unbranched C₁-C₄-alkyl substituents specified under W, R⁹ to R¹⁰ are, for example, a methyl, ethyl, n-propyl, n-butyl group, and the branched C₃-C₄-alkyl groups are an isopropyl, isobutyl, sec-butyl, tert-butyl group.

The alkyl groups may optionally be mono- to or polysubstituted by halogen atoms, e.g. fluorine, chlorine or bromine.

The saturated unbranched C₁-C₆-alkyl substituents specified under R¹-R⁷ are, for example, a methyl, ethyl, n-propyl, n-butyl, n-pentyl, n-hexyl group, and the branched C₃-C₆-alkyl groups are an isopropyl, isobutyl, sec-butyl, tert-butyl, isopentyl, neopentyl, 2-methylpentyl, 2,2-dimethylbutyl, 2,3-dimethylbutyl group.

The alkyl groups may optionally be mono- to polysubstituted by halogen atoms (e.g. fluorine, chlorine or bromine), cyano, hydroxyl, amino, carboxyl groups or an optionally mono- or polysubstituted 5-6-membered aryl or heteroaryl radical. Examples of a 5-6-membered aryl radical include the following: cyclopentadienyl, phenyl.

The 5-6-membered heteroaryl groups may be a pyridyl, pyrimidyl, furanyl, thiophenyl, oxazolyl, isoxazolyl, thiazolyl, pyrrolyl, pyrazolyl, pyrazinyl, pyridazinyl, triazolyl, tetrazolyl or imidazolyl group bonded via one of the substitutable positions.

The C₂-C₆-alkenyl substituents in R²-R⁵ or the C₂-C₄-alkenyl substituents in X, R⁹-R¹⁰ are each straight-chain or branched, meaning, for example, the following radicals:

Vinyl, allyl, homoallyl, (E)-but-2-enyl, (Z)-but-2-enyl, pent-4-enyl, (E)-pent-3-enyl, (Z)-pent-3-enyl, (E)-pent-2-enyl, (Z)-pent-2-enyl, 2-methylvinyl, 3-methylbut-3-enyl, 2-methylbut-3-enyl, (E)-2-methylbut-2-enyl, (Z)-2-methylbut-2-enyl, 2-ethylprop-2-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, 1-methylpent-4-enyl, (E)-1-methylpent-3-enyl, (Z)-1-methylpent-3-enyl, 1-ethylbut-3-enyl, (E)-1-methylpent-2-enyl, (Z)-1-methylpent-2-enyl.

The alkenyl groups may optionally be mono- to trisubstituted by halogen atoms (e.g. fluorine, chlorine or bromine), cyano, carboxyl groups, or an optionally mono- or polysubstituted 5-6-membered aryl or heteroaryl radical. Examples of a 5-6-membered aryl radical include the following: cyclopentadienyl, phenyl.

The 5-6-membered heteroaryl groups may be a pyridyl, pyrimidyl, furanyl, thiophenyl, oxazolyl, isoxazolyl, thiazolyl, pyrrolyl, pyrazolyl, pyrazinyl, pyridazinyl, triazolyl, tetrazolyl or imidazolyl group bonded via one of the substitutable positions.

The C₂-C₆-alkynyl substituents in R²-R⁵, and the C₂-C₄-alkynyl substituents in X and R⁹-R¹⁰ are each straight-chain or branched, meaning, for example, the following radicals: ethynyl, prop-1-ynyl, but-1-ynyl, but-2-ynyl, pent-1-ynyl, hex-1-ynyl.

The alkynyl groups may optionally be monosubstituted by halogen atoms (e.g. fluorine, chlorine or bromine), cyano, carboxyl groups or an optionally mono- or polysubstituted 5-6-membered aryl or heteroaryl radical.

Examples of a 5-6-membered aryl radical include the following: cyclopentadienyl, phenyl.

The 5-6-membered heteroaryl groups may be a pyridyl, pyrimidyl, furanyl, thiophenyl, oxazolyl, isoxazolyl, thiazolyl, pyrrolyl, pyrazolyl, pyrazinyl, pyridazinyl, triazolyl, tetrazolyl or imidazolyl group bonded via one of the substitutable positions.

Halogen is understood to mean the following: fluorine, chlorine, bromine, iodine.

The C₃-C₁₀-cycloalkyl in R¹-R⁷ and the C₃-C₆-cycloalkyl in R⁹-R¹⁰ are understood to mean monocyclic alkyl rings such as cyclopropyl, cyclobutyl, cyclopentyl, cyclohexyl or cycloheptyl, or cyclooctyl, but also bicyclic rings, for example decahydronaphthalene, tricyclic rings or bridged rings, for example adamantanyl.

The cycloalkyl groups may optionally be mono- to disubstituted by halogen atoms (e.g. fluorine, chlorine or bromine), and also by cyano, hydroxyl, amino, carboxyl groups.

The 5-12-membered mono- or bicyclic aryl or heteroaryl radical which is unsubstituted or optionally mono- or polysubstituted, and is specified in R¹, R²-R⁵, R⁶-R⁷ may be understood to mean 5-12-membered ring systems which, instead of the carbon, may contain one or more identical or different heteroatoms, such as oxygen, nitrogen or sulfur, in the ring, may be mono- or bicyclic and may additionally each be benzofused.

Examples of a 5-12-membered mono- or bicyclic aryl radical include the following: cyclopentadienyl, phenyl, tropyl, cyclooctadienyl, indenyl, naphthyl, azulenyl, biphenyl.

The 5,12-membered mono- or bicyclic heteroaryl groups may be a pyridinyl, pyrimidinyl, quinolinyl, isoquinolinyl, phthalazinyl, quinazolinyl, quinoxalinyl, cinnolinyl, benzofuranyl, benzothiophenyl, 1,3-benzodioxolyl, benzimidazolyl 2,1,3-benzothiadiazolyl, indolyl, furanyl, thiophenyl, oxazolyl, isoxazolyl, thiazolyl, pyrrolyl, pyrazolyl, pyrazinyl, pyridazinyl, triazolyl, tetrazolyl or imidazolyl group bonded via one of the substitutable positions.

The 5-6-membered aryl or heteroaryl radical which may be unsubstituted or optionally mono- or trisubstituted and is specified in R⁹ to R¹⁰ is understood to mean 5-6-membered ring systems which, instead of the carbon, may contain one or more identical or different heteroatoms, such as oxygen, nitrogen or sulfur, in the ring and are bonded to the skeleton via one of the possible bonding sties.

Examples of a 5-6-membered mono- or bicyclic aryl radical include the following: cyclopentadienyl, phenyl.

The 5-6-membered heteroaryl groups may be a pyridinyl, pyrimidinyl, furanyl, thiophenyl, oxazolyl, isoxazolyl, thiazolyl, pyrrolyl, pyrazolyl, pyrazinyl, pyridazinyl, tetrazolyl, triazolyl or imidazolyl group bonded via one of the substitutable positions.

The unsubstituted 8-12-membered fused (hetero)arylcycloalkyl radical which has been described in R¹ and is unsubstituted or optionally mono- or polysubstituted may, for example, but not exclusively, be an indanyl, 2,3-dihydro-1H-indolyl, 2,3-dihydrobenzofuranyl, 2,3-dihydrobenzo[b]thiophenyl, benzol[1,3]dioxolyl, tetralinyl, 1,2,3,4-tetrahydroquinolinyl or chromanyl group bonded via one of the substitutable positions.

The 3-8-membered ring which can be formed by ring closure of R⁶ and R⁷ or R⁹ and R¹⁰ may be a cycloalkyl or a nitrogen-containing heterocycle. Examples of a 3-8-membered cycloalkyl ring include, for example, the following: cyclopropyl, cyclobutyl, cyclopentyl, cyclohexyl or cycloheptyl, cyclooctyl.

Examples of a 3-8-membered nitrogen-containing heterocycle include, for example, the following: aziridinyl, azetidinyl, pyrrolidinyl, piperidinyl, piperazinyl, morpholinyl, azepanyl, [1,4]-diazepanyl.

The free alcohols of the inventive compounds may also be present as esters and are thus prodrugs of the physiological compounds of the general formula I which, in the organism, metabolize to compounds of the general formula I.

Suitable compounds are listed, for example, in Hans Bundgaard (ed.), Design of Prodrugs, Elsevier, Amsterdam 1985.

When an acidic function is present, suitable salts are the physiologically compatible salts of organic and inorganic bases, for example the readily soluble alkali metal and alkaline earth metal salts, and also N-methylglucamine, dimethylglucamine, ethylglucamine, lysine, 1,6-hexadiamine, ethanolamine, glucosamine, sarcosine, serinol, tris(hydroxymethyl)aminomethane, aminopropanediol, Sovak base, 1-amino-2,3,4-butanetriol.

When a basic function is present, useful methods for the formation of physiologically compatible salts of the inventive compounds of the general formula I are methods known to those skilled in the art; useful inorganic acids include hydrochloric acid, hydrobromic acid, sulfuric acid and phosphoric acid, nitric acid; useful carboxylic acids include acetic acid, propionic acid, hexanoic acid, octanoic acid, decanoic acid, oleic acid, stearic acid, maleic acid, fumaric acid, succinic acid, benzoic acid, ascorbic acid, oxalic acid, salicylic acid, tartaric acid, citric acid, lactic acid, glycolic acid, malic acid, mandelic acid, cinnamic acid, glutamic acid, aspartic acid; useful sulfonic acids include methanesulfonic acid, ethanesulfonic acid, toluenesulfonic acid, benzenesulfonic acid and naphthalenesulfonic acid.

Preference is given to the compounds of the general formula I where
W is hydrogen or a methyl group,
X is a (CH₂)ₙ group where n=0-4, a C₂-C₄-alkenyl group, a C₂-C₄-alkynyl group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
   a C₁-C₆-alkyl radical which is unsubstituted or optionally substituted,
   a C₃-C₁₀-cycloalkyl radical which is unsubstituted or optionally substituted,
   an 8-12-membered fused (hetero)arylcycloalkyl radical which is unsubstituted or optionally substituted,
R²-R⁵ are each independently hydrogen, halogen, cyano, or an OR⁶, OC(O)R⁶, S(O)ₙR⁶ where n=0, 1, 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
   a C₁-C₆-alkyl group which may be unsubstituted or optionally substituted,
   a C₃-C₁₀-cycloalkyl ring which may be unsubstituted or optionally substituted,
   a C₂-C₆-alkenyl or C₂-C₆-alkynyl group which may be unsubstituted or optionally substituted,
   a 5-12-membered mono- or bicyclic aryl or heteroaryl ring which may be unsubstituted or optionally substituted,
R⁶, R⁷ are each hydrogen, a C₁-C₆-alkyl group, a C₃-C₁₀-cycloalkyl ring, a 5-12-membered mono- or bicyclic aryl or heteroaryl ring, where the alkyl, cycloalkyl and (het)aryl groups may be unsubstituted or optionally substituted, or
R⁶, R⁷ together form a 3-8-membered ring.

Likewise preferred are the compounds of the general formula I where
W is hydrogen or a methyl group,
X is a (CH₂)ₙ group where n=0-4, a C₂-C₄-alkenyl group, a C₂-C₄-alkynyl group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
   where the substituents may be selected from the group of halogen, R⁶, —OR⁶, —OC(O)R⁶, —S(O)ₙR⁶ where n=0, 1, 2, —SO₂NHR⁶, —SO₂NHC(O)R⁶, NR⁶R⁷, —NHC(O)R⁶, NO₂, —CN, —CO₂—R⁶, —C(O)—N—R⁶R⁷, —C(O)R⁶, —C(OH)R⁶R⁷,
   a C₁-C₆-alkyl radical which may be unsubstituted or optionally up to pentafluorinated,
   an unsubstituted C₃-C₁₀-cycloalkyl radical, or
   an unsubstituted 8-12-membered fused (hetero)aryl cycloalkyl radical,
R² is hydrogen,
R³-R⁵ are each independently hydrogen, halogen, cyano, or an OR⁶, OC(O)R⁶, S(O)NR⁶ where n=0, 1, 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
   a C₁-C₆-alkyl group which is unsubstituted or optionally substituted or unsubstituted,
   a C₃-C₁₀-cycloalkyl ring which is unsubstituted or optionally substituted,
   a C₂-C₆-alkenyl or C₂-C₆-alkynyl group which is unsubstituted or optionally substituted,
   a 5-12-membered mono- or bicyclic aryl or heteroaryl ring which is unsubstituted or optionally mono- or polysubstituted,
      where the substituents may be selected from the group of
         halogen,
         C₁-C₄-alkyl which may be unsubstituted or substituted,
         OR⁹, —OC(O)R⁹, —S(O)ₙR⁹ where n=0, 1, 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹, —C(OH)R⁹R¹⁰,
   where the 5-12-membered mono- or bicyclic aryl or heteroaryl ring may, for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl, benzimidazolyl group,
R⁶, R⁷ are each hydrogen,
   a C₁-C₆-alkyl group which may be unsubstituted or optionally up to pentahalogenated,
   a C₃-C₁₀-cycloalkyl radical,
   a 5-12-membered mono- or bicyclic aryl or heteroaryl ring which is unsubstituted or optionally mono- or polysubstituted,
      where the substituents may be selected from the group of
         halogen,
         cyano,
         R⁹, —OR⁹, —OC(O)R⁹, —S(O)ₙR⁹ where n=0, 1, 2, —SO₂NHR⁹, NR⁹R¹⁰, —NHC(O)R⁹, —CO₂—R⁹, —C(O)—N—R⁹R¹⁰,
   where the 5-12-membered mono- or bicyclic aryl or heteroaryl ring may, for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ together form a 3-8-membered ring,
R⁹, R¹⁰ are each independently hydrogen,
   a C₁-C₄-alkyl group which may be unsubstituted or optionally up to pentafluorinated,
   a C₂-C₄-alkenyl group which may be unsubstituted or optionally up to trifluorinated,
   a C₂-C₄-alkynyl group which may be unsubstituted or optionally monofluorinated,
   a C₃-C₆-cycloalkyl group,
   a 5-6-membered aryl or heteroaryl ring which may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl ring, and where the 5-6-membered aryl or heteroaryl ring may be unsubstituted or optionally up to disubstituted by fluorine, chlorine, trifluoromethyl, or
R⁹, R¹⁰ together form a 3-8-membered ring.

Likewise preferred are compounds of the general formula I where
W is hydrogen or a methyl group
X is a (CH₂)ₙ group where n=0-2, a —CH═CH— group, a —C≡C group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
   where the substituents may be selected from the group of halogen, R⁶, OR⁶, —OC(O)R⁶, —S(O)ₙR⁶ where n=0, 1, 2, —SO₂NHR⁶, —SO₂NHC(O)R⁶, NR⁶R⁷, —NHC(O)R⁶—NO₂, —CN, —CO₂—R⁶, —C(O)—N—R⁶R⁷, —C(O)R⁶, —C(OH)R⁶R⁷,
   an unsubstituted C₃-C₁₀-cycloalkyl radical,
   or an unsubstituted 8-12-membered fused (hetero)arylcycloalkyl radical,
R² is hydrogen,
R³-R⁵ are each independently hydrogen, halogen, cyano, or an OR⁶, OC(O)R⁶, S(O)NR⁶ where n=0, 1, 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
   a C₁-C₆-alkyl group which may be unsubstituted or optionally substituted,
   an unsubstituted C₃-C₁₀-cycloalkyl ring,
   a C₂-C₆-alkenyl group which may be unsubstituted or optionally substituted,
   a C₂-C₆-alkynyl group, which may be unsubstituted or optionally monosubstituted,
   a mono- or bicyclic 5-6-membered aryl or heteroaryl ring which is unsubstituted or optionally mono- or polysubstituted,
      where the substituents may be selected from the group of
         halogen,
         C₁-C₄-alkyl which may be unsubstituted or up to pentahalogenated or else substituted by —OH, —CN, —CO₂H,
         OR⁹, —OC(O)R⁹, —S(O)ₙR⁹ where n=0, 1, 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹, —C(OH)R⁹R¹⁰,
   where the 5-6-membered aryl or heteroaryl ring may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl group,
R⁶, R⁷ are each hydrogen, a C₁-C₄-alkyl group which may be unsubstituted or optionally up to pentahalogenated,
   a C₃-C₆-cycloalkyl radical,
   a 5-6-membered aryl or heteroaryl ring which is unsubstituted or optionally mono- or polysubstituted,
      where the substituents may be selected from the group of
         halogen,
         cyano,
         R⁹, —OR⁹, —OC(O)R⁹, —S(O)ₙR⁹ where n=0, 1, 2, —SO₂NHR⁹, NR⁹R¹⁰, —NHC(O)R⁹, —CO₂—R⁹, —C(O)—N—R⁹R¹⁰,
   where the 5-6-membered aryl or heteroaryl ring may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ are each independently hydrogen,
   a C₁-C₄-alkyl group which may be unsubstituted or optionally up to pentafluorinated,
   a C₂-C₄-alkenyl group which may be unsubstituted or optionally up to trifluorinated,
   a C₂-C₄-alkynyl group which may be unsubstituted or optionally monofluorinated,
   a C₃-C₆-cycloalkyl group,
   a 5-6-membered aryl or heteroaryl ring which may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl ring, which may be unsubstituted or optionally up to disubstituted by fluorine, chlorine, trifluoromethyl, or
R⁹, R¹⁰ together form a 3-8-membered ring.

The following compounds according to the present invention are very particularly preferred:
N-[1-(7-Bromo-phthalazin-1-yl)-piperidin-4-yl]-2,3-dichloro-benzamide
N-[1-(6-Bromo-phthalazin-1-yl)-piperidin-4-yl]-2,3-dichloro-benzamide
N-[1-(7-Bromo-4-methyl-phthalazin-1-yl)-piperidin-4-yl]-2,3-dichloro-benzamide
N-[1-(6-Bromo-4-methyl-phthalazin-1-yl)-piperidin-4-yl]-2,3-dichloro-benzamide
2,3-Dichloro-N-(1-phthalazin-1-yl-piperidin-4-yl)-benzamide.

The present invention provides for the use of the inventive compounds for the production of medicaments which comprise at least one of the compounds of formula I.

The present invention likewise provides medicaments which comprise the inventive compounds with suitable formulation and carrier substances.

Compared with the known prostaglandin E₂ ligands, the novel EP₂ agonists and antagonists are notable for greater selectivity and stability.

The present invention provides medicaments for treatment and prophylaxis of disorders which include fertility disorders, infectious diseases, cancer, viral infections, cardiovascular disorders, elevated intraocular pressure, glaucoma, disorders of the skeletal system, angiogenic disorders, abnormalities of uterine contraction, pain, neuroinflammatory disorders, immunomodulatory infections and nephrological disorders.

Fertility disorders are understood to mean disorders leading to no ovulation taking place, to nidation of a fertilized oocyte not taking place and no decidualization taking place; infectious diseases are understood to mean diseases caused by unicellular parasites; cancer is understood to mean solid tumors and leukemia; viral infections are understood to mean, for example, cytomegalus infections, hepatitis, hepatitis B and C and HIV disorders; immunomodulatory infections are understood to mean, for example, bird flu; cardiovascular disorders are understood to mean ischemic reperfusion disorder, stenoses, arterioscleroses and restenoses; angiogenic disorders are understood to mean, for example, endometriosis and fibrosis; elevated intraocular pressure is understood to mean glaucoma; abnormalities of uterine contraction are understood to mean, for example, menstrual complaints; disorders of the skeletal system are understood to mean osteoporosis; neuroinflammatory disorders are understood to mean multiple sclerosis, Alzheimer's disease, pain; and nephrological disorders are understood to mean glomerulonephritis.

The present invention likewise provides medicaments for treatment and prophylaxis of the disorders listed above, which comprise at least one compound of the general formula I, and also medicaments comprising suitable formulation and carrier substances.

For use of the compounds according to the invention as medicaments, they are converted to the form of a pharmaceutical product which, in addition to the active ingredient, comprises pharmaceutical, organic or inorganic inert carrier materials suitable for enteral or parenteral administration, for example water, gelatin, gum arabic, lactose, starch, magnesium stearate, talc, vegetable oils, polyalkylene glycols etc. The pharmaceutical products may be in solid form, for example as tablets, coated tablets, suppositories, capsules, in semisolid form, for example as ointments, creams, gels, suppositories, emulsions, or in liquid form, for example as solutions, suspensions or emulsions.

If appropriate, they comprise excipients intended to function, for example, as fillers, binders, disintegrants, lubricants, solvents, solubilizers, masking flavors, dye, emulsifiers. Excipient types in the context of the invention are, for example, saccharides (mono-, di-, tri-, oligo- and/or polysaccharides), fats, waxes, oils, hydrocarbons, anionic, nonionic, cationic natural, synthetic or semisynthetic surfactants. If appropriate, they additionally comprise excipients such as preservatives, stabilizers, wetting agents or emulsifiers; salts to alter the osmotic pressure or buffers.

The present invention likewise provides these pharmaceutical products.

Aerosol solutions are appropriately produced for inhalation.

Particularly suitable for oral administration are tablets, coated tablets or capsules with talc and/or carbohydrate carriers or binders, for example lactose, corn starch or potato starch. Use is also possible in liquid form, for example as fluid to which a sweetener is added where appropriate. For oral administration of such compounds, clathrates are likewise also suitable; examples include the clathrates with alpha-, beta-, gamma-cyclodextrin, or else beta-hydroxypropylcyclodextrin.

Sterile, injectable, aqueous or oily solutions are used for parenteral administration. Solutions for injection or suspensions, especially aqueous solutions of the active compounds in polyethoxylated castor oil are particularly suitable.

Suppositories, tampons or intrauterine devices, for example, are suitable and customary for vaginal administration.

For intraarticular injection, it is possible to use appropriately formulated crystal suspensions.

For intramuscular injection, it is possible to use aqueous and oily solutions for injection or suspensions and corresponding depot preparations.

For rectal administration, it is possible to use the novel compounds in the form of suppositories, capsules, solutions (for example in the form of enemas) and ointments both for systemic and for local therapy.

For pulmonary administration of the novel compounds, they can be used in the form of aerosols and inhalations.

For local administration on eyes, the external auditory canal, middle ear, nasal cavity and paranasal sinuses, the novel compounds may be used as drops, ointments and tinctures in appropriate pharmaceutical formulations.

For topical administration, formulations in gels, ointments, greasy ointments, creams, pastes, powder, milk and tinctures are possible. The dosage of the compounds of the general formula I in these formulations should be 0.01%-20% in order to achieve a sufficient pharmacological effect.

Carrier systems which can also be used are surface-active excipients such as salts of bile acids or animal or vegetable phospholipids, but also mixtures thereof, and liposomes or constituents thereof.

The dosage of the active ingredients may vary depending on the route of administration, age and weight of the patient, nature and severity of the disorder to be treated and similar factors. The treatment can be effected in single doses or as a large number of doses over a prolonged period. The daily dose is 0.5-1000 mg, preferably 50-200 mg, it being possible for the dose to be given as a single dose to be administered once or divided into 2 or more daily doses.

The above-described formulations and administration forms likewise form part of the subject matter of the present invention.

The inventive compounds can be administered by any conventional method including oral and parenteral methods, for example by subcutaneous or intramuscular injections. Enteral, parenteral, vaginal and oral administration likewise form part of the subject matter of the present invention.

The inventive compounds of the general formula I bind to the EP₂ receptor and have agonistic or antagonistic action. It can be determined by an agonism test (see example 1.2.1 of the biological examples) or by an antagonism test (see example 1.2.2 of the biological examples) whether agonistic or antagonistic action is present.

Antagonists are understood to mean those molecules which bind to their corresponding receptors and typically compete with the naturally occurring ligand of the receptor for the binding to the receptor and which inhibit the initiation of the signal transduction pathway coupled to the receptor.

Receptor antagonists typically bind selectively to their particular receptor and not to other receptors. They normally have a higher binding affinity than the natural ligand. Even though antagonists which have a higher affinity for the receptor than the natural ligand are preferred, it is likewise possible to use antagonists with a lower affinity.

The antagonists preferably bind reversibly to their corresponding receptors.

The EP₂ receptor antagonist has a preferential affinity for the EP₂ receptor over any other EP receptor. The antagonism is measured in the presence of the natural agonist (PGE₂).

Agonists are understood to mean those molecules which bind to their corresponding receptors and typically compete with the naturally occurring ligand of the receptor for the binding to the receptor and which stimulate the initiation of the signal transduction pathway coupled to the receptor. Agonists may also promote the binding of the natural ligand.

Receptor agonists typically bind selectively to their particular receptor and not to other receptors. They normally have a higher binding affinity than the natural ligand. Even though agonists which have a higher affinity for the receptor than the natural ligand are preferred, it is likewise possible to use agonists with a lower affinity.

The agonists preferably bind reversibly to their corresponding receptors.

Agonists are tested via the initiation of the signal transduction and/or physiological action mediated the corresponding receptor.

Ligands refer to the compounds or low molecular weight substances which bind to a receptor. Their binding is typically reversible. The binding of a ligand to the corresponding receptor activates or inactivates the signal transduction pathway coupled to the receptor. In this manner, the ligand imparts its intracellular action. Ligands are understood to mean agonists and antagonists of a receptor.

The substance according to example 2 exhibits no inhibition in the cellular agonism test (EC₅₀>19 μM), but good efficacy in the antagonism test (IC₅₀=200 nM).

The present invention likewise provides for the use of the inventive substances as EP₂ receptor agonists for the treatment of disorders caused by disruptions in the signal transduction chain in which the EP₂ receptor is involved, for example pain, endometriosis, fertility disorders, and which are likewise suitable for fertility control.

The inventive compounds of the general formula I have profertile action. In the preovulatory antral follicle, the oocyte is surrounded by cumulus cells which form a dense ring of cells around the oocyte. After the peak of the lutenizing hormone (LH peak), a series of processes is activated and leads to a great morphological change in this ring of cumulus cells. The cumulus cells form an extracellular matrix which leads to so-called cumulus expansion (Vanderhyden et al. Dev Biol. 1990 August; 140(2):307-317). This cumulus expansion is an important part of the ovulatory process and of the subsequent possibility of fertilization.

In cumulus expansion, prostaglandins, and here prostaglandin E₂ whose synthesis is induced by the LH peak, are of crucial significance. Prostanoid EP₂ knockout mice (Hizaki et al., Proc Natl Acad Sci USA 1999 Aug. 31; 96(18):10501-6) exhibit markedly reduced cumulus expansion and severe subfertility, which demonstrates the significance of the prostanoid EP₂ receptor for this process.

The inventive substances have inhibitory effects in cumulus expansion tests.

The present invention provides for the use of the inventive substances for fertility control.

While the EP₂ receptor antagonist AH 6809 suppresses the expansion of the cumulus by only about 20% at a concentration of 100-200 μM, a 35% suppression of cumulus expansion can be achieved at one tenth of the concentration in the presence of the substance according to example 2. In these tests, the test substances compete with the natural EP₂ receptor agonist PGE₂.

The present invention provides for the use of the inventive substances for the inhibition of cumulus expansion and hence of ovulation and fertilization for contraception.

Prostaglandins play an important role in angiogenesis (Sales, Jabbour, 2003, Reproduction 126, 559-567).

Endometriosis is a chronic disorder caused by impairments of the blood vessels. About 10% of women regularly suffer from chronic bleeding during menstruation caused by changes in the blood vessels of the endometrium. In addition, structural differences in the blood vessels have been observed, for example incomplete formation of the smooth muscle cell layer (Abberton et al., 1999, Hum. Reprod. 14, 1072-1079). Since blood loss during menstruation is controlled partly by the constriction of the blood vessels, it is obvious that the defects in the smooth muscle structure make a substantial contribution to the bleeding.

The present invention provides for the use of the substances of the general formula I for the treatment of endometriosis.

Prostaglandins play an important role in uterus contraction; excessively strong contractions are responsible for menstrual pains (Sales, Jabbour, 2003, Reproduction 126, 559-567).

The present invention provides for the use of the substances of the general formula I for the treatment of menstrual pains.

Prostaglandins play an important role in the development and course of various cancers (S. W. Han, Biochemical and Biophysical Research Communications 314 (2004) 1093-1099; S.-H. Chang; Cancer Research 65 (2005); 4496-9; M. D. Castellone, Science 310 (2005) 1504-1510).

The present invention provides for the use of the substances of the general formula I for the treatment and prevention of cancers.

Prostaglandins also play an important role in the processes which counteract osteoporosis. The present invention therefore provides for the use of the inventive substances for the treatment of osteoporosis.

Reinold et al. (J. Clin. Invest. 115, 673-679 (2005)) describe PGE₂ receptors of the EP₂ subtype as the key signaling elements in inflammatory hyperalgesia. Mice which no longer have this receptor (EP2^{-/-}) experience no spinal inflammatory pain. There are indications that inflammatory, enhanced pain sensitivity can be treated by modulating EP₂ receptors in a controlled manner.

The present invention provides for the use of the inventive substances for the treatment of inflammatory hyperalgesia.

Where the preparation of the starting compounds is not described, these can be prepared in a known manner or analogously to known compounds or processes described here. It is likewise possible to perform all reactions described here in parallel reactors or by means of combinatorial techniques.

The salts are prepared in a customary manner by admixing a solution of the compound of the formula I with the equivalent amount or an excess of a base or acid, which may be in solution, and removing the precipitate or working up the solution in a customary manner.

The invention thus also relates to medicaments based on the compounds of the general formula I and the customary excipients or carriers.

The inventive compounds of the general formula I can be prepared as described in the examples. Proceeding from the 1-chlorophthalazines of the general formula IV, it is possible to prepare the inventive compounds of the general formula I by reacting with N-piperidin-4-yl(het)arylamides of the general formula V by processes known to those skilled in the art. It is likewise possible to prepare the inventive compounds of the general formula I by converting 1-chlorophthalazines of the general formula IV to aminopiperidinylphthalazines of the general formula III and then formula II by processes known to those skilled in the art. By an analogous procedure using reagents homologous to the reagents described in the examples, it is possible to obtain the further compounds of the general formula I.

The R²-R⁵ radicals of the compounds of the general formula I obtained in this way can be converted further by methods known to those skilled in the art to various functional groups and hence further compounds of the general formula I.

For example, a bromide can be replaced by methods known to those skilled in the art by means of palladium(0)-catalyzed reactions by an aryl or heteroaryl ring, a substituted alkene or alkyne, amine or a cyano group.

A carboxyl function or cyano group functioning as R²-R⁵, or an amine can, for example, be converted by methods known to those skilled in the art to esters and amides of the general formula I.

It is likewise possible, for example, to convert ester functions or a cyano group in compounds of the general formula I, after reduction to the aldehyde, by methods known to those skilled in the art, to further olefins or secondary alcohols substituted by alkyl or aryl radicals. It is likewise possible to convert a cyano group in compounds of the general formula I, by methods known to those skilled in the art, to ketones which are substituted by alkyl or aryl radicals and can then be reduced to the corresponding secondary alcohols or else, by methods known to those skilled in the art, converted to tertiary alcohols substituted by alkyl or aryl radicals.

The exemplary reactions just described of the R²-R⁵ radicals in the inventive compounds of the general formula I can be performed in the same manner by a person skilled in the art on compounds of the general formula II and III. The compounds of the general formula II and III thus obtained can then be converted to those of the formula I as described.

The 1-chlorophthalazines of the general formula IV used to prepare the inventive compounds of the general formula I can be prepared by processes known to those skilled in the art depending on the W radical. In the case that W=hydrogen, this is done, for example, proceeding from the phthalides of the general formula VIII or phthalic anhydrides of the general formula XI via the phthalazines of the general formulae VII to give those of the general formula IV. In the case that W=C₁-C₃-alkyl, this is done for example proceeding from the phthalic anhydrides of the general formula XI via the 3-hydroxy-3-alkyl-3H-isobenzofuran-1-ones of the general formula IX and the 3-alkylidene-3H-isobenzofuran-1-ones of the general formula X to give the phthalazines of the general formulae VII and further to those of the general formula IV.

The N-piperidin-4-yl(het)arylamides of the general formula V used to prepare the inventive compounds of the general formula I can be prepared by methods known to those skilled in the art proceeding from tert-butyl 4-aminopiperidine-1-carboxylate via the tert-butyl 4-{[(het)arylcarbonyl]amino}piperidine-1-carboxylate of the general formula VI.

Frequently used abbreviations:

EA ethyl acetate
Cx cyclohexane
equiv. equivalents
DMAP 4-dimethylaminopyridine

The examples which follow serve to illustrate the invention in detail:

### General Procedure for the Synthesis of the Aminopiperidinylphthalazines I

The corresponding N-piperidin-4-yl(het)arylamide V (1 equiv.) is initially charged in n-butanol (10 ml/mmol), admixed with 1 equiv. of the appropriate 1-chlorophthalazine IV, with 2 equiv. of triethylamine and with 0.1 equiv. of DMAP, and stirred under reflux until the reaction is complete or has stopped. After cooling to room temperature, the reaction mixture is admixed with EA, washed with sat. sodium chloride solution and concentrated on a rotary evaporator. The purification is effected by column chromatography on silica gel with a Cx/EA eluent and gives rise to the phthalazines 1. According to this general reaction procedure, the following compounds, for example, were synthesized: 1-5.

The examples synthesized are characterized by means of analytical HPLC-MS [Method 1: Hy Purity Elite C18 column (250×4.6 mm, 5 μm), gradient: 10% acetonitrile in 10 mM ammonium formate (pH 7.7) (20 min), flow: 1.0 ml/min, MS: (M+H)⁺; Method 2: Hypersil 120 ODS column (150×4.0 mm, 5 μm), gradient: 10% acetonitrile in 10 mM ammonium formate (pH 7.7) on 100% acetonitrile (20 min), flow: 1.0 ml/min, MS: (M+H)⁺].

| | | | MW | | RT (min., |
|---|---|---|---|---|---|
| Example | Structure | Name | calc. | MW | method) |
| 1 | 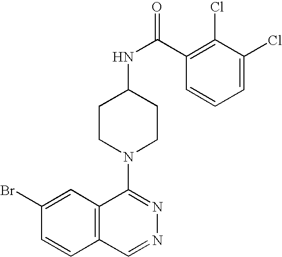 | N- [1-(7-Bromo-phthalazin-1-yl)-piperidin-4-yl]-2,3-dichloro-benzamide | 480.191 | 481 | 16.01 (1) |
| 2 | 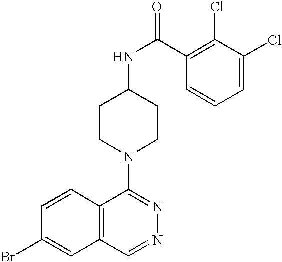 | N-[1-(6-Bromo-phthalazin-1-yl)-piperidin-4-yl]-2,3-dichloro-benzamide | 480.191 | 481 | 15.60 (1) |
| 3 | 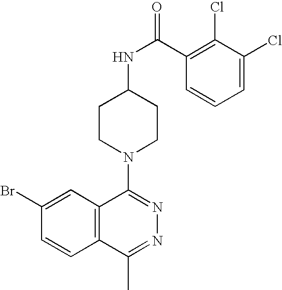 | N-[1-(7-Bromo-4-methyl-phthalazin-1-yl)-piperidin-4-yl]-2,3-dichloro-benzamide | 494.217 | 495 | 16.44 (1) |
| 4 | 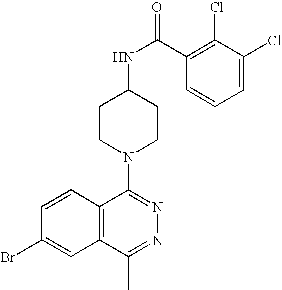 | N-[1-(6-Bromo-4-methyl-phthalazin-1-yl)-piperidin-4-yl]-2,3-dichloro-benzamide | 494.217 | 495 | 16.26 (1) |
| 5 | 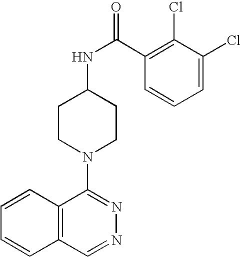 | 2,3-Dichloro-N-(1-phthalazin-1-yl-piperidin-4-yl)-benzamide | 401.30 | 401 | 10.93 (2) |

### Synthesis Schemes

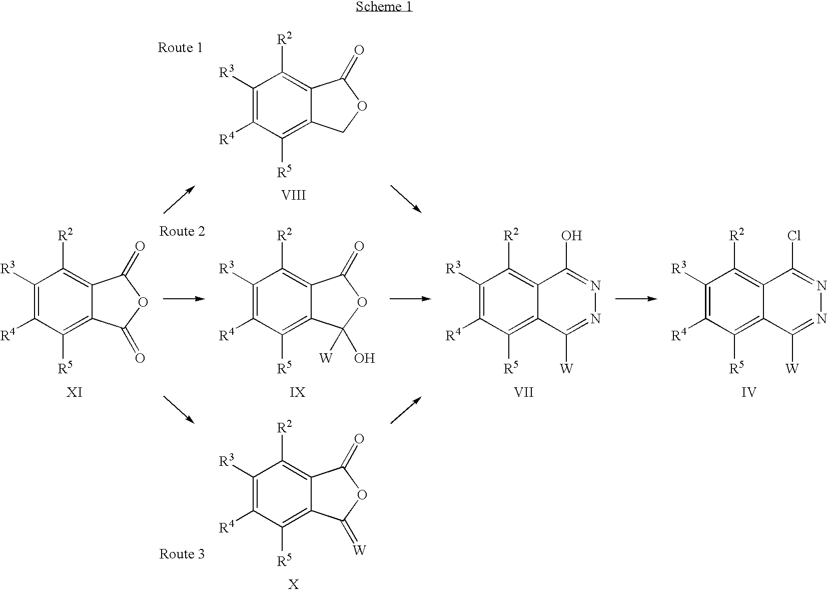

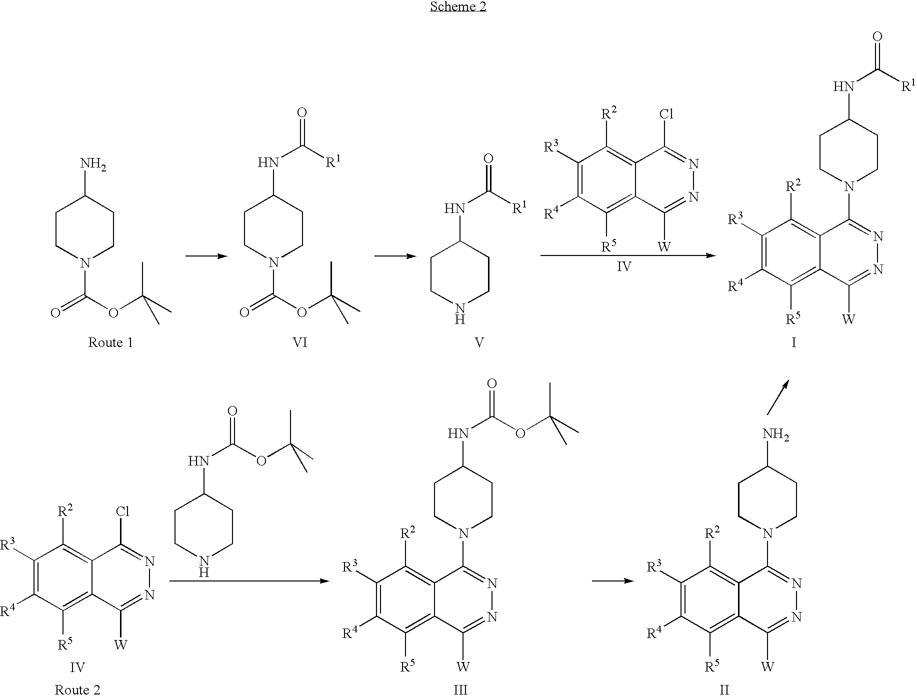

### BIOLOGICAL EXAMPLES

### 1. Detection of the Antagonism of the Human Prostaglandin E₂ (Subtype EP₂) Receptor Signal

### 1.1 Principle of Detection

The binding of PGE₂ to the EP₂ subtype of the human PGE₂ receptor induces activation of membrane-associated adenylate cyclases and leads to the formation of cAMP. In the presence of the phosphodiesterase inhibitor IBMX, cAMP which has accumulated due to this stimulation and been released by cell lysis is employed in a competitive detection method. In this assay, the cAMP in the lysate competes with cAMP-XL665 for binding of an Eu cryptate-labeled anti-cAMP antibody.

This results, in the absence of cellular cAMP, in a maximum signal which derives from coupling of this antibody to the cAMP-XL665 molecule. After excitation at 337 nm, this results in a FRET (fluorescence resonance energy transfer)-based, long-lived emission signal at 665 nm (and at 620 nm). The two signals are measured in a suitable measuring instrument with a time lag, i.e. after the background fluorescence has declined. Any increase in the low FRET signal caused by prostaglandin E₂ addition (measured as well ratio change=emission_{665 nm}/emission_{620 nm}*10 000) shows the effect of antagonists.

### 1.2. Detection Method

### 1.2.1 Antagonism Assay (Data for Each Well of a 384-Well Plate):

The substance solutions (0.75 μl) containing 30% DMSO are introduced into an assay plate and dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # I-7018), and then 15 μl, thereof are transferred into a media-free cell culture plate which has been washed with KRSB shortly beforehand.

After preincubation at room temperature (RT) for 30 minutes, 5 μl of a 4×PGE₂ solution (11 nM) are added, and incubation is carried out in the presence of the agonist at RT for a further 60 min (volume: ˜20 μl) before the reaction is then stopped by adding 5 μl of lysis buffer and incubated at RT for a further 20 min (volume: ˜25 μl). The cell lysate is then transferred into a measuring plate and measured in accordance with the manufacturer's information (cyclic AMP kit Cisbio International #62AMPPEC).

### 1.2.2 Agonism Assay (Data for Each Well of a 384-Well Plate):

The substance solutions (0.75 μl) containing 30% DMSO are introduced into an assay plate and dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # I-7018), and then 15 μl thereof are transferred into a media-free cell culture plate which has been washed with KRSB shortly beforehand.

After incubation at room temperature (RT; volume: ˜15 μl) for 60 minutes, the reaction is then stopped by adding 5 μl of lysis buffer and incubated at RT for a further 20 min (volume: ˜20 μl). The cell lysate is then transferred into a measuring plate and measured in accordance with the manufacturer's information (cyclic AMP kit Cisbio International #62AMPPEC).

### 2. The EP₂ Subtype of the PGE₂ Receptor and the Preovulatory Cumulus Expansion

### 2.1. Background:

In the preovulatory antral follicle, the oocyte is surrounded by cumulus cells which form a dense ring of cells around the oocyte. After the LH peak (lutenizing hormone), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140(2):307-317). This cumulus expansion is an important component of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96(18):10501-6.) show a markedly reduced cumulus expansion and severe subfertility, demonstrating the importance of the prostanoid EP₂ receptor for this process.

### 2.2 Cumulus Expansion Assay In Vitro

Folliculogenesis is induced in immature female mice (strain: CD1 (ICR) from Charles River) at an age of 14-18 days by a single dose (intraperitoneally) of 10 I.U. of PMSG (Pregnant Mare Serum Gonadotropin; Sigma G-4877, Batch 68H0909). 47-50 hours after the injection, the ovaries are removed and the cumulus-oocyte complexes are removed. The cumulus complex is not yet expanded at this stage.

The cumulus-oocyte complexes are then incubated with prostaglandin E₂ (PGE₂) (1 μM), vehicle control (ethanol) or test substances for 20-24 hours. Medium: alpha-MEM medium with 0.1 mM IBMX, pyruvates (0.23 mM), glutamines (2 mM), pen/strep (100 IU/ml pen. and 100 μg/ml strep.) and HSA (8 mg/ml). Cumulus expansion is then established through the division into four stages (according to Vanderhyden et al. Dev Biol. 1990 August; 140(2):307-317).

TABLE 1
Examples of the biological efficacy of the inventive compounds
(measured by means of cAMP antagonism assay):

| Substance | | |
|---|---|---|
| according to | Agonism | |
| example | [ED₅₀, μM] | Antagonism [IC₅₀, μM] |
| 1 | >19 | 0.3 |
| 2 | >19 | 0.2 |
| 3 | >19 | 0.7 |
| 4 | >19 | 0.5 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 06 090 159.2, filed Sep. 7, 2006, and U.S. Provisional Application Ser. No. 60/842,679, filed Sep. 7, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of the general formula I 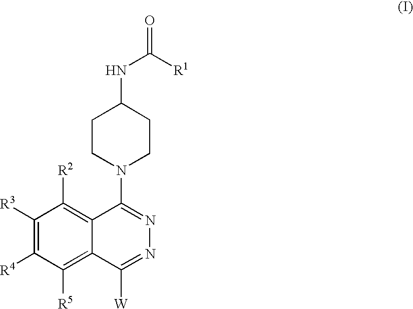
where
W is hydrogen or a C₁-C₄-alkyl group,
X is a (CH₂)ₙ group where n=0-4, a C₂-C₄-alkenyl group, a C₂-C₄-alkynyl group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
a C₁-C₆-alkyl group which is unsubstituted or optionally substituted,
a C₃-C₁₀-cycloalkyl radical which may be unsubstituted or optionally substituted
an 8-12-membered fused (hetero)arylcycloalkyl radical which is unsubstituted or optionally substituted.
R²-R⁵ are each independently hydrogen, halogen, cyano, or an OR⁶, OC(O)R⁶, S(O)NR⁶ where n=0, 1, 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
a C₁-C₆-alkyl group which may be unsubstituted or optionally substituted,
a C₃-C₁₀-cycloalkyl ring which may be unsubstituted or optionally substituted,
a C₂-C₆-alkenyl or C₂-C₆-alkynyl group which may be unsubstituted or optionally substituted,
a 5-12-membered mono- or bicyclic aryl or heteroaryl ring which may be unsubstituted or optionally substituted,
R⁶, R⁷ are each hydrogen, a C₁-C₆-alkyl group, a C₃-C₁₀-cycloalkyl ring, a 5-12-membered mono- or bicyclic aryl or heteroaryl ring, where the alkyl, cycloalkyl and (het)aryl groups may be unsubstituted or optionally substituted, or
R⁶, R⁷ together form a 3-8-membered ring, and the isomers, salts and the cyclodextrin clathrates thereof.

2. A compound as claimed in claim 1, where
W is hydrogen or a methyl group,
X is a (CH₂)ₙ group where n=0-4, a C₂-C₄-alkenyl group, a C₂-C₄-alkynyl group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
a C₁-C₆-alkyl group which is unsubstituted or optionally substituted,
a C₃-C₁₀-cycloalkyl radical which is unsubstituted or optionally substituted,
an 8-12-membered fused (hetero)arylcycloalkyl radical which is unsubstituted or optionally substituted.
R²-R⁵ are each independently hydrogen, halogen, cyano, or an OR⁶, OC(O)R⁶, S(O)NR⁶ where n=0, 1, 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
a C₁-C₆-alkyl group which may be unsubstituted or optionally substituted,
a C₃-C₁₀-cycloalkyl ring which may be unsubstituted or optionally substituted,
a C₂-C₆-alkenyl or C₂-C₆-alkynyl group which may be unsubstituted or optionally substituted,
a 5-12-membered mono- or bicyclic aryl or heteroaryl ring which may be unsubstituted or optionally substituted,
R⁶, R⁷ are each hydrogen, a C₁-C₆-alkyl group, a C₃-C₁₀-cycloalkyl ring, a 5-12-membered mono- or bicyclic aryl or heteroaryl ring, where the alkyl, cycloalkyl and (het)aryl groups may be unsubstituted or optionally substituted, or
R⁶, R⁷ together form a 3-8-membered ring.

3. A compound as claimed in claim 1, where
W is hydrogen or a methyl group,
X is a (CH₂)ₙ group where n=0-4, a C₂-C₄-alkenyl group, a C₂-C₄-alkynyl group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
where the substituents may be selected from the group of halogen, R⁶, —OR⁶, —OC(O)R⁶, —S(O)ₙR⁶ where n=0, 1, 2, —SO₂NHR⁶, —SO₂NHC(O)R⁶, NR⁶R⁷, —NHC(O)R⁶, NO₂, —CN, —CO₂—R⁶, —C(O)—N—R⁶R⁷, —C(O)R⁶, —C(OH)R⁶R⁷,
a C₁-C₆-alkyl radical which may be unsubstituted or optionally up to pentafluorinated,
an unsubstituted C₃-C₁₀-cycloalkyl radical, or
an unsubstituted 8-12-membered fused (hetero)arylcycloalkyl radical,
R² is hydrogen,
R³-R⁵ are each independently hydrogen, halogen, cyano,
or an OR⁶, OC(O)R⁶, S(O)ₙR⁶ where n=0, 1, 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
a C₁-C₆-alkyl group which is unsubstituted or optionally substituted or unsubstituted,
a C₃-C₁₀-cycloalkyl ring which is unsubstituted or optionally substituted,
a C₂-C₆-alkenyl or C₂-C₆-alkynyl group which is unsubstituted or optionally substituted,
a 5-12-membered mono- or bicyclic aryl or heteroaryl ring which is unsubstituted or optionally mono- or polysubstituted,
where the substituents may be selected from the group of
halogen,
C₁-C₄-alkyl which may be unsubstituted or substituted,
OR⁹, —OC(O)R⁹, —S(O)ₙR⁹ where n=0, 1, 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹, —C(OH)R⁹R¹⁰,
where the 5-12-membered mono- or bicyclic aryl or heteroaryl ring may, for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl, benzimidazolyl group,
R⁶, R⁷ are each hydrogen,
a C₁-C₆-alkyl group which may be unsubstituted or optionally up to pentahalogenated,
a C₃-C₁₀-cycloalkyl radical,
a 5-12-membered mono- or bicyclic aryl or heteroaryl ring which is unsubstituted or optionally mono- or polysubstituted,
where the substituents may be selected from the group of
halogen,
cyano,
R⁹, —OR⁹, —OC(O)R⁹, —S(O)ₙR⁹ where n=0, 1, 2, —SO₂NHR⁹, NR⁹R¹⁰, —NHC(O)R⁹, —CO₂—R⁹, —C(O)—N—R⁹R¹⁰,
where the 5-12-membered mono- or bicyclic aryl or heteroaryl ring may, for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ together form a 3-8-membered ring,
R⁹, R¹⁰ are each independently hydrogen,
a C₁-C₄-alkyl group which may be unsubstituted or optionally up to pentafluorinated,
a C₂-C₄-alkenyl group which may be unsubstituted or optionally up to trifluorinated,
a C₂-C₄-alkynyl group which may be unsubstituted or optionally monofluorinated,
a C₃-C₆-cycloalkyl group,
a 5-6-membered aryl or heteroaryl ring which may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl ring, and where the 5-6-membered aryl or heteroaryl ring may be unsubstituted or optionally up to disubstituted by fluorine, chlorine, trifluoromethyl, or
R⁹, R¹⁰ together form a 3-8-membered ring.

4. A compound according to claim 1, where
W is hydrogen or a methyl group
X is a (CH₂)ₙ group where n=0-2, a —CH═CH— group, a —C≡C group,
R¹ is a 5-12-membered mono- or bicyclic aryl or heteroaryl ring, a 5-12-membered mono- or bicyclic O-aryl or O-heteroaryl ring, S-aryl or S-heteroaryl ring, N-aryl or N-heteroaryl ring, where the rings may be unsubstituted or optionally mono- to trisubstituted,
where the substituents may be selected from the group of halogen, R⁶, —OR⁶, —OC(O)R⁶, —S(O)ₙR⁶ where n=0, 1, 2, —SO₂NHR⁶, —SO₂NHC(O)R⁶, NR⁶R⁷, —NHC(O)R⁶, —NO₂, —CN, —CO₂—R⁶, —C(O)—N—R⁶R⁷, —C(O)R⁶, —C(OH)R⁶R⁷,
an unsubstituted C₃-C₁₀-cycloalkyl radical,
or an unsubstituted 8-12-membered fused (hetero)arylcycloalkyl radical,
R² is hydrogen,
R³-R⁵ are each independently hydrogen, halogen, cyano, or an OR⁶, OC(O)R⁶, S(O)NR⁶ where n=0, 1, 2, SO₂NHR⁶, SO₂NHC(O)R⁶, NR⁶R⁶, NHC(O)R⁶, CH₂NR⁶R⁷, CH₂NHC(O)R⁶, C(OH)R⁶R⁷, C(O)R⁶, CO₂R⁶, C(O)NR⁶R⁷ group,
a C₁-C₆-alkyl group which may be unsubstituted or optionally substituted, an unsubstituted C₃-C₁₀-cycloalkyl ring,
a C₂-C₆-alkenyl group which may be unsubstituted or optionally substituted,
a C₂-C₆-alkynyl group, which may be unsubstituted or optionally monosubstituted,
a mono- or bicyclic 5-6-membered aryl or heteroaryl ring which is unsubstituted or optionally mono- or polysubstituted,
where the substituents may be selected from the group of
halogen,
C₁-C₄-alkyl which may be unsubstituted or up to pentahalogenated or else substituted by —OH, —CN, —CO₂H,
OR⁹, —OC(O)R⁹, —S(O)ₙR⁹ where n=0, 1, 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹, —C(OH)R⁹R¹⁰,
where the 5-6-membered aryl or heteroaryl ring may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl group,
R⁶, R⁷ are each hydrogen, a C₁-C₄-alkyl group which may be unsubstituted or optionally up to pentahalogenated,
a C₃-C₆-cycloalkyl radical,
a 5-6-membered aryl or heteroaryl ring which is unsubstituted or optionally mono- or polysubstituted,
where the substituents may be selected from the group of
halogen,
cyano,
R⁹, —OR⁹, —OC(O)R⁹, —S(O)ₙR⁹ where n=0, 1, 2, —SO₂NHR⁹, NR⁹R¹⁰, —NHC(O)R⁹, —CO₂—R⁹, —C(O)—N—R⁹R¹⁰,
where the 5-6-membered aryl or heteroaryl ring may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ are each independently hydrogen,
a C₁-C₄-alkyl group which may be unsubstituted or optionally up to pentafluorinated,
a C₂-C₄-alkenyl group which may be unsubstituted or optionally up to trifluorinated,
a C₂-C₄-alkynyl group which may be unsubstituted or optionally monofluorinated,
a C₃-C₆-cycloalkyl group,
a 5-6-membered aryl or heteroaryl ring which may, for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl ring, which may be unsubstituted or optionally up to disubstituted by fluorine, chlorine, trifluoromethyl, or
R⁹, R¹⁰ together form a 3-8-membered ring.

5. A compound as claimed in claim 1, selected from a group which comprises the following compounds:
N-[1-(7-Bromo-phthalazin-1-yl)-piperidin-4-yl]-2,3-dichloro-benzamide
N-[1-(6-Bromo-phthalazin-1-yl)-piperidin-4-yl]-2,3-dichloro-benzamide
N-[1-(7-Bromo-4-methyl-phthalazin-1-yl)-piperidin-4-yl]-2,3-dichloro-benzamide
N-[1-(6-Bromo-4-methyl-phthalazin-1-yl)-piperidin-4-yl]-2,3-dichloro-benzamide
2,3-Dichloro-N-(1-phthalazin-1-yl-piperidin-4-yl)-benzamide

6. The use of the compounds as claimed in claim 1 for producing medicaments which comprise at least one of the compounds of the formula I.

7. A medicament as claimed in claim 6 comprising suitable formulation and carrier substances.

8. The use of the medicament as claimed in claim 6, characterized in that the medicament is used for treatment and prophylaxis of disorders.

9. The use as claimed in claim 8 for treatment and prophylaxis of disorders connected to the EP₂ receptor.

10. The use as claimed in claim 8 for treatment and prophylaxis of fertility disorders.

11. The use as claimed in claim 8 for treatment and prophylaxis of menstrual pains.

12. The use as claimed in claim 8 for treatment and prophylaxis of endometriosis.

13. The use of the compounds as claimed in claim 1 for modulation of the EP₂ receptor.

14. The use as claimed in claim 8 for treatment and prophylaxis of pain.

15. The use of the compounds as claimed in claim 1 and of the medicaments for fertility control.

16. The use as claimed in claim 8 for treatment and prophylaxis of osteoporosis.

17. The use as claimed in claim 8 for treatment and prophylaxis of cancer.

18. The use of the compounds of the general formula I as claimed in claim 1 in the form of a pharmaceutical preparation for enteral, parenteral, vaginal and oral administration.

19. A method of fertility control comprising administering a compound of claim 1.
